(19) United States
(12) Patent Application Publication
(10) Pub. No.: US 2026/0183170 A1
(43) Pub. Date: Jul. 2, 2026

(54) MEANS OF ATTACHING A DISPOSABLE DRAINAGE BAG FOR UROLOGICAL, GYNECOLOGICAL AND OTHER SURGICAL PROCEDURES

(71) Applicant: Kyra Medical, Inc, Northborough, MA (US)

(72) Inventors: Jesse DRAKE, Northborough, MA (US); Ryan CUNNIFF, Hopkinton, MA (US); Jeremy Macpherson, Medfield, MA (US); Justin MCCARTHY, Boxford, MA (US)

(21) Appl. No.: 17/683,884

(22) Filed: Mar. 1, 2022

Related U.S. Application Data
(60) Provisional application No. 63/207,489, filed on Mar. 3, 2021.

(51) Int. Cl. A61M 1/00; A61G 13/10

(52) U.S. Cl. CPC A61G 13/101; A61G 13/102; A61M 1/69

(57) ABSTRACT

In one aspect, a drainage bag system configured to be coupled to a medical procedural table is disclosed, which comprises a drainage bag, a band attached to and supporting the drainage bag, said band having at least one protuberance positioned at least one end thereof, and at least one mounting clamp including a receiving slot configured to receive said at least one protuberance of the band. The clamp has a receiving channel configured to receive a surgical table accessory rail or an accessory mount arm thereof, and the clamp has a threaded member configured to secure the clamp to the surgical table accessory or an accessory mount arm.

### RELATED APPLICATION

The present application claims priority under 35 U.S.C. § 119(e) to U.S. provisional application no. 63/207,489, filed on Mar. 3, 2021, which is herein incorporated by reference in its entirety.

### FIELD

The present disclosure relates generally to drainage systems for use in medical procedures and particularly for use in medical procedures in which lithotomy stirrups are employed.

### BACKGROUND

When performing a surgical procedure, and more particularly a urological surgical or a gynecological surgical procedure, clinicians may use fluids such as sterile saline for irrigation of the operative site. It is necessary to collect such fluids and record the amount of the fluid used during the procedure to ensure patient safety. Allowing such fluids to puddle or collect on the floor could present a hazard to the clinician and surgical team members. Many times, such procedures are performed with the patient in the supine position and with the patient's legs positioned up and away from the operative site near the perinium. In order to obtain access to the operative site the patient's legs may be positioned in lithotomy stirrups to optimize access to the operative site. Such stirrups are attached to the surgical table by clamps which are mounted at the distal end of the surgical table side rails.

Drainage bags, more particularly urology drainage bags, that collect fluids during the surgical procedure, are known. There are reusable flexible band systems which connect with reusable clamps that mount to the distal ends of side rails of surgical tables. Reusable flexible bands allow mounting of a disposable drainage bag that then collects fluids during the procedure. There are also systems that consist of a disposable drainage bag mounted to a flexible band which in turn connects to the surgical table by means of a reusable rail mounted clamp. These reusable clamps are mounted at the distal end of the surgical table side rail. In the art there are also disposable drainage bag systems which consist of a disposable drainage bag mounted to a flexible band to which is attached disposable clamps which connect to the distal end of surgical table side rails. These disposable clamps will fit only one size of surgical table rails. The advantage of the completely disposable drainage bag system described above is that the clamps do not need to be cleaned and sterilized after each procedure and then stored for eventual re-use.

However, the clamps of such a system, and all conventional clamps used in the current art described in this application, occupy a portion of the distal end of the surgical table rail. According to the current art when disposable or reusable clamps of these systems are used with lithotomy stirrups the clamps that mount the stirrups must be moved proximally towards the patient's head since the disposable clamps are mounted at the distal end of the surgical table rail. Not mounting the lithotomy stirrups at the distal end of the surgical table rails may prevent the stirrup's proximal locking joint from being positioned at the patient's hip joint as recommended by all stirrup manufacturers.

If the proximal locking joint of the stirrup is not mounted at the patient's hip joint an increase in the stress on the patient's hip may occur, which may lead to possible injury or post procedural pain.

### SUMMARY

This disclosure is generally directed to surgical drainage bag systems that are disposable (i.e., they can be discarded after each use). In some embodiments, such a surgical drainage bag system can include a disposable clamp, a disposable flexible band to which is attached a disposable drainage bag drape, all of which can be discarded after use. This system allows the user to attach the system to a surgical table and then discard the system after each use.

As discussed in detail below, a surgical drainage bag system according to the present teachings can allow the safe positioning of a patient since it allows the stirrup mounting clamp to be positioned at the distal end of the side rail of a surgical table. Where stirrups are not used, a surgical drainage bag system according to the present teachings allows for mounting the disposable clamps onto the distal end of the surgical table rails to secure the disposable drainage bag system.

Surgical table side rails to which accessories are attached, including drainage bag systems, come in a variety of sizes including differing widths and heights and include, but are not limited to US, EU and UK surgical table side rails. In the current art both the reusable clamps and disposable clamps of the conventional drainage bag systems will fit only one size of surgical table rails. This means that a facility with varying tables with rail sizes would have to stock a number of conventional types of drainage bag systems each with differing sized clamps. The current invention allows one disposable clamp to be engaged onto a variety of rail sizes meaning that hospitals with a variety of tables with differing rail sizes need procure only one type of drainage bag system.

In many urological and gynecological procedures, which require a fluid collection system, other accessories, equipment and surgical aids may be attached via surgical rail clamps to the side rails present on most surgical and procedural tables. Lithotomy stirrups are used to lift and hold the legs away from the operative site are but one example of commonly used surgical equipment accessories and/or surgical aids. Lithotomy stirrups when attached to the surgical table side rails via surgical rail clamps help provide the clinician access to the operative site. These lithotomy stirrups frequently employ gas filled cylinders which provide an upward force reducing the stress or strain on the clinician when lifting and positioning the patient's legs. The gas cylinders are typically attached at a distal mount point on the stirrup support rod and at a proximal mounting lug on a mount plate which is at about the equal level of the surgical table rail.

In some embodiments, a surgical drainage bag system according to the present teachings can include a flexible band and a disposable drainage bag drape along with a disposable clamp (which may be attached to the surgical table side rail), all of which can be discarded after each use plus an accessory mount mechanism that allows securing the disposable drainage bag, the flexible band and the clamp to equipment or accessories configured to be mounted onto a medical table, such as procedural or surgical tables.

In many embodiments, the disposable clamp allows the acceptance of a wide variety of sizes of surgical table rails obviating the need to stock multiple drainage bag systems with differing sized clamps to fit such differing rail sizes. The accessory mount mechanism may be attached to certain surgical accessories or equipment used during medical procedures in order to secure the disposable drainage bag system for use. Such an accessory mount for the disposable drainage bag system obviates the need for rail mounting the drainage bag system when certain surgical accessories are used, thus creating more space at the distal end of the surgical table rail. This additional space at the distal end of the surgical table rail may allow a lithotomy stirrup's proximal locking joint to be properly mounted at the location of a patient's hip joint, which in turn reduces the risk of creating stress at the patient's hip joint, that could lead to injury during the procedure and/or post procedural patient hip pain.

In embodiments, a disposable drainage bag system may include a disposable drainage bag, a flexible band coupled to and supporting the disposable drainage bag and a disposable clamp that is configured to accept a surgical table side rail or an accessory mount arm, which may be sized similar to a surgical table side rail.

The disposable clamp may include a channel to accept a surgical table rail, or an accessory mount arm, and a band acceptance slot, sized to accept the flexible band, on the other side of the disposable clamp. In some embodiments the band acceptance slot may have indentations and/or holes at pre-determined positions within the band acceptance slot that may be sized to receive detents, buttons, protuberances and protrusions that may be formed into the flexible band. In some embodiments, an upper ledge and a lower ledge may be formed into the disposable clamp body. These ledges may each have a stabilizer extension formed therein that are substantially rigid and substantially parallel to the lower surface and upper surfaces of the lower and upper ledges. The term “substantially rigid” is defined herein as deflecting less than 0.10 inch while substantially parallel means not deviating more than 10 degrees from the referenced surface. In some embodiments, the disposable clamp may be formed of a plastic, such as polyethylene, polyurethane, polycarbonate, polyethylene terephthalate, or acrylonitrile-butadiene-styrene (ABS).

In some embodiments a disposable clamp for coupling a flexible band and a drainage bag to a surgical table side rail or accessory mount arm is disclosed. The disposable clamp may include a rail acceptance channel on one side thereof for accepting the side rail of a surgical table or an accessory mount arm. In some embodiments the rail acceptance channel may be formed by the upper ledge and lower ledge of the disposable clamp while each ledge may have lips formed into the distal edges of the upper and lower ledges of the disposable clamps. The clamp may have a body including a proximal side in some embodiments. In some embodiments, the proximal side of the clamp may have a notch cut or formed therein. The acceptance rail channel may be shaped and sized to fit onto a surgical table rail or an accessory mount arm.

In some embodiments the lower surface of the upper ledge may have a chase cut into it allowing the acceptance of differing sizes of accessory rails; in particular rails that may have a width narrower, or a height larger, than the rail acceptance channel of the disposable clamp. In other embodiments upper ledge chase can fit onto at least one fin formed into accessory mount arms to stabilize the disposable clamp when so mounted. In some embodiments, the lower ledge can be formed to include a threaded hole that can receive a threaded knob which can be engaged into and through the threaded hole. In some embodiments the threaded hole is offset from the center of the clamp body and is aligned on the centerline of the chase formed into the lower surface of the upper ledge of the disposable clamp. In some embodiments, a threaded screw can be engaged through a threaded hole in lower platform and a surgical rail may be inserted into upper ledge chase thus engaging the surgical table rail with upper ledge chase. In some embodiments the lower surface of the lip of the upper ledge is lower than the notch cut onto the upper ledge of the proximal clamp surface forming one side of the rail acceptance channel. In other embodiments, a threaded screw can be engaged through a threaded hole in the lower platform engaging the clamp with an accessory mount arm. In other embodiments, the upper ledge may be formed to include a threaded hole that can receive a threaded knob which can be engaged into and through the threaded hole. In some embodiments, the lower ledge may include a distal lip and also include an extension stabilizer that protrudes from the lower ledge opposite the side of the disposable clamp's band acceptance slot. In one embodiment the upper ledge of the clamp may include a lip on its distal side and also may have an engagement surface, which is lower than a notch in the proximal clamp wall and may also have a stabilizer extension that protrudes from the ledge on the side opposite the disposable clamp's band acceptance slot. In such an embodiment, the lips formed on the upper ledge may protrude distally and down and away from the upper surface of this ledge to assist in engaging the surgical table rail or accessory mount arms. In some embodiments the lips formed on the lower ledge may protrude distally and up and away from the lower surface of the lower ledge to assist in engaging the surgical table rail or accessory mount arm. In some embodiments, the proximal side of the clamp body with notch, the lower upper ledge and the lower ledge, the upper and lower edge lips and the upper and lower stabilizer extensions of the ledges may form a monolithic body formed, e.g., of plastic.

The proximal side of the clamp body with notch, the lower surface of upper ledge, the lips of the upper ledge including an engagement surface and the lower ledge with lips, and the upper and lower stabilizer extensions of the ledges make up the rail acceptance channels into which a surgical table rail or accessory mount arm may be engaged. In some embodiments the lower surface of the upper ledge may have a chase cut into it to accept rails that are narrower and/or taller than the width of the rail acceptance channel. In other embodiments the components of the surgical rail acceptance channel may include separate plastic or metal pieces or a combination of metal and plastic parts that can be assembled, e.g., via press fit, welded or glued together. In yet other embodiments the disposable clamp body, ledges, lips and extension stabilizers can be formed of metal.

The disposable clamp may be engaged onto a table surgical table rail or accessory mount arm using the clamp rail acceptance channel. In some embodiments the upper portions of surgical table rails are narrower and/or taller than rail acceptance channel and can fit into the upper ledge chase. In other embodiments the upper surface chase can be fitted onto a fin formed into upper surface of an accessory mount arm. The threaded knob can then be inserted and engaged into the threaded hole in the lower ledge such that the distal portion of the threaded knob in turn engages the surgical table rail or an accessory mount arm thus securing the disposable clamp in place. In other embodiments the threaded knob can be engaged into, and through, the threaded hole pushing against rails narrower and/or taller than rail engagement channel and engaging them into the upper ledge chase thus securing this rail to the disposable clamp.

In some embodiments the disposable clamp side opposite the rail acceptance channel may have a flexible band acceptance slot formed in it. The flexible band acceptance slot may have at least one indentation in one embodiment. The at least one indentation can have a variety of shapes including, but not limited to, rectilinear, square, cuboidal, round, oblong, hexagonal, round or obolid or formed in other shapes. In other embodiments the clamp rail channel may have at least one through hole. The at least one through hole can have a variety of shapes including, but not limited to rectilinear, square, cuboidal, round/circular, obolid, oblong or hexagonal. In other embodiments the band acceptance slot can have at least two indentations in it and each of the indentations can be of the same or differing shapes or sizes including, but not limited to, the following shapes: rectilinear, square, cuboidal, round/circular, obloid, oblong or hexagonal. In other embodiments the disposable clamp band acceptance slot may have at least two through holes. The at least two through holes can have a variety of shapes including, but not limited to the following: rectilinear, square, cuboidal, round/circular, obloid, oblong or hexagonal.

In some embodiments the clamp side opposite the rail acceptance channel may have a band acceptance slot formed in it. The band acceptance slot may have at least one indentation in one embodiment. The at least one indentation can be formed to accept protuberances or protrusions that are serrated or barbed or other polygonal shapes. In other embodiments the clamp rail channel may have at least one through hole or indentation. The at least one through hole or indentation can be formed to accept protuberances that are serrated or barbed protrusions or of other polygonal shapes. In other embodiments the clamp band slot can have at least two indentations or through holes in it and each of the at least two through holes or at least two indentations can be of the same or differing shapes or sizes in any combination that might accept barbed or serrated protuberances or other polygonal shapes. In other embodiments the at least one through hole or indentation can be formed to accept a variety of shapes including but not limited to barbed or serrated shaped protrusions.

In some embodiments the clamp side opposite the rail acceptance channel may have a band acceptance slot formed in it. In some embodiments the band acceptance slot may have at least one indentation and at least one through hole. The at least one indentation and the at least one through hole can be formed to accept protuberances that are serrated or barbed protrusions or otherwise shaped and in any combination of shapes. The clamp band slot can have at least one indentation and at least one through hole in it and each of the at least one through hole or the at least one indentation can be of the same or differing shapes or sizes in any combination that might accept barbed or serrated protuberances or shapes in any combination.

In some embodiments the surgical drainage bag system can include a disposable drainage drape bag, a flexible band, a disposable clamp and an accessory mount mechanism. This accessory mount mechanism may be secured to an accessory or other equipment used in surgical procedures, in particular lithotomy stirrups, which are configured to be attached to a procedural or surgical table in some embodiments. In one embodiment the accessory mount mechanism can be discarded after use. In other embodiments the accessory mount mechanism may be semi-permanently attached to an accessory or other equipment configured to attach to a procedural or surgical table in particular lithotomy stirrups. This accessory mount mechanism may be formed or molded of plastics parts in one embodiment. In other embodiments it may be formed or made of metal. In yet other embodiments it may be made or formed of a combination of plastic and metal parts. It is contemplated in some embodiments that the accessory mount mechanism may be formed as a monolithic part of either plastic or metal. In other embodiments it can be made of two or more separate plastic or metal pieces that may be press fit using indentations and counter-pressed protuberances or protrusions or held together by other mechanical means such as an attachment rod. In some embodiments the accessory mount mechanism may include a monolithic accessory mount clamp formed of plastic or of metal with at least two holes formed in the structure and with at least one of the holes being threaded, plus a separate spacer formed of plastic or made of metal with a through hole and an attachment rod threaded at one end with a flat head on the opposite end.

The accessory mount mechanism, in some embodiments, may include a horseshoe or “u” shaped accessory mount clamp with holes formed in two of its arms, a spacer with a through hole and an attachment rod threaded at one end with a flat head at its opposite end. In some embodiments the spacer is “t” shaped while in other embodiments it is shaped as a rectilinear block. In yet other embodiments it can be round or semi-circular in shape. The accessory mount clamp is shaped and sized so that it fits snugly and closely around the outside surfaces of a stirrup gas cylinder mount lug of a lithotomy stirrup while the spacer is attached to the mount accessory clamp. There are differing sized proximal mount lugs for gas cylinders for various makes of stirrups. In some embodiments, there are differing sized accessory mount clamps and spacers that will accommodate and fit closely and snuggly around the outside surfaces of various sizes of gas cylinder gas mount lugs for each make of stirrup.

In some embodiments the accessory mount clamp can include an upper arm, a lower arm and a semi-circular section. The upper arm, the lower arm and the semi-circular section of the accessory mount clamp is designed to fit closely and snuggly with the outside surface of the stirrup gas cylinder mount lug. In some embodiments the upper arm, lower arm and semi-circular sections of the accessory mount clamp are formed or molded as a monolithic part out of plastic or alternatively formed or made of metal. In yet other embodiments the sections are plastic or metal and are press fit or glued together using indentations with slots to accept barbed or serrated protrusions or protuberances. In one embodiment the upper arm is formed with a threaded hole that can accept a threaded rod while the lower arm is formed with a hole that is opposite the corresponding hole in the upper arm. In other embodiments the lower arm has a threaded hole while the upper arm has a non-threaded hole which is directly opposite the hole of the upper arm. In some embodiments a mount arm is formed and centered on the distal edge of the semi-circular section and protrudes outward at substantially right angles from this section. The term substantially right angles is defined as plus or minus 5 degrees from 90 degrees. The mount arm is the size of a surgical table rail. In some embodiments a fin which is sized to fit into the upper ledge chase of disposable clamp is formed into the upper surface of accessory mount arm. A spacer formed or molded from plastic or made from metal, almost equal in length to twice the inner radius of the semi-circular section of the accessory mount clamp in some embodiments, is made or formed of plastic or of metal. The spacer has a through hole running from its top through to its bottom in some embodiments. The spacer fits on part of the surface of the stirrup gas spring mount lug and the through hole formed in the spacer that lines up with the holes formed in the upper and lower arms of the accessory mount clamp. In one embodiment an attachment rod (machined or formed of metal, plastic or suitable materials) with one threaded end and a flat head on the opposite end is passed through the lower arm of the accessory mount clamp and into and through the through hole in the spacer. The threaded end of the rod is designed to fit into, and engage with, the threaded hole in the upper arm of the accessory mount clamp in one embodiment.

In some embodiments a spacer rod (machined or formed of metal) with one threaded end and a flat head on the opposite end is passed through the hole in the upper arm of the accessory mount clamp into and through the through hole in the spacer. The threaded end of the rod is engaged into and with the threaded hole in the lower arm of the accessory mount clamp thus securing the accessory mount clamp to the surfaces of stirrup gas cylinder mount lug. In some embodiments the spacer is rectangular in shape. In other embodiments it is “T” shaped with a flange. In some embodiments the “T” shaped flange has side having a semi-circular cut while in other embodiments this “T” flange has a rectilinear cut out. In other embodiments the spacer is circular, obloid or semi-circular in shape and may have a flange of varying shapes including but not limited to square, circular or hexagonal. The spacer, secured by the threaded rod to the upper and lower arms of the accessory mount clamp, also fits snuggly to a surface of the stirrup gas cylinder mount lug.

It is contemplated that mount arm with fin in upper surface that protrudes from the distal side of the semicircular section of the accessory mount clamp is formed or molded as part of the monolithic plastic or metal structure of the accessory mount clamp in one embodiment. In other embodiments the mount arm and fin may be formed of separate pieces of plastic or metal and connected to the accessory mount clamp by means of press fit connections or gluing/welding or any combinations thereof. The mount arm can be sized to conform to the size of a surgical table rail in some embodiments.

In some embodiments the accessory mount clamp may be attached to the stirrup gas cylinder mount lug by fitting the accessory mount clamp's upper arm, lower arm and semi-circular sections around the surface or surfaces of stirrup gas cylinder mount lug. By passing the attachment rod through the upper arm of the accessory mount clamp and into and through the through hole in the spacer it can be engaged into the threaded hole of the lower arm of the accessory mount clamp thus securing the spacer to the accessory mount clamp and in turn securing the accessory mount clamp to the stirrup gas cylinder mount lug. The side of the disposable clamp with the rail acceptance channel can be slid onto the mount arm with fin of the accessory mount clamp with the upper ledge chase fitting round the mount arm fin. The upper and lower stabilizer extension arms of the disposable rail clamp and the fit of the mount arm fin onto upper ledge chase help stabilize the disposable clamp resisting any torsion forces. The threaded knob of the disposable clamp can be engaged into the threaded hole in lower ledge of the disposable clamp securing it to the mount arm of the accessory mount clamp in some embodiments. The flexible band with the attached drainage bag drape can then be secured to the disposable clamp.

The accessory mount mechanism, in some embodiments, may include a horseshoe or “u” shaped accessory mount clamp with upper, lower and curved arms with holes formed in its upper and lower arms, a spacer with a through hole and an attachment rod threaded at one end with a flat head at its opposite end. In some embodiments, the spacer is “t” shaped while in other embodiments it is shaped as a block. In yet other embodiments it can be round or semi-circular in shape. In some embodiments the spacer may have a rectilinear or circular flange and the flange may have a semi-circular or rectilinear cutout. The accessory mount clamp is sized so that it fits snugly and closely around the surface or surfaces of a protrusion or extension of a surgical accessory or surgical equipment with such extensions being between about 0.25 to about 4 inches in diameter (for circular protrusions) or longest cross section width (in rectilinear protrusions) being between about 0.25 to about 4 inches and protrusions extending out from ½ inches up to eight inches in length from the surface of the equipment or accessory in some embodiments. The spacer, when attached to the mount accessory clamp fits, snuggly around the proximal surfaces of the protrusion of the surgical accessory or equipment when such a protrusion is between 0.25 to 4 inches in diameter or cross sectional width of from about 0.25 to about 4 inches and extending outward from the surface of the accessory or equipment up to eight inches in length in some embodiments.

The flexible band may be formed of commonly available plastic or other suitable materials. The flexible band may have at least one prong formed into each of its ends in one embodiment. The at least one prong may have at least one protrusion or protuberance that can be formed into differing shapes. In some embodiments the at least one protuberance or protrusion in the prong may be, but is not limited to, the following shapes: rectilinear, square, cuboidal, round, obolid, oblong, hexagonal or round in any combination but sized to fit in the at least one indentation in the flexible band acceptance slot. In other embodiments the shapes of the at least one protuberance or protrusion in the at least one prongs may be serrated or barbed in shape and in any combination but sized to fit into the at least one indentation in the flexible band acceptance slot. In some embodiments the flexible band may have at least one notch cut into its ends forming at least one prong with at least one protuberance on the prong of the flexible band.

The flexible band may be formed of commonly available plastic or other suitable materials. The flexible band may have at least two prongs formed into each of its ends in some embodiments. The at least two prongs may have at least one protrusion or protuberances each that can be formed into differing shapes. In some embodiments the at least one protuberance or protrusion formed in the at least two prongs may be, but is not limited to, the following shapes: rectilinear, square, cuboidal, round, obolid, oblong, hexagonal or round in any combination but sized to fit in the at least two indentations in the flexible band acceptance slot. In other embodiments the shapes of the at least one protuberance or protrusion in the at least two prongs may be serrated or barbed in shape and in any combination but sized to fit into the at least two indentations in the flexible band acceptance slot. In some embodiments the flexible band may have at least one notch cut into its ends forming at least two prongs with at least one protuberance on the at least two prongs of the flexible band.

In some embodiments the distance between the at least one protuberance on the differing prongs of the flexible band is slightly larger than the height of the disposable band acceptance slot formed into the side of the disposable clamp. The term “slightly large” is defined herein as being between about 0.1 to about 0.15 inches larger. When the flexible band is fitted into the disposable band acceptance slot the at least two prongs are depressed by the force exerted on the at least two prongs as they are engaged into the band acceptance slot. As the end of the flexible band is slid into the acceptance band slot the at least two protuberances engage with the at least two indentations or through holes in the flexible band acceptance slots thus connecting the flexible band with the disposable clamp.

The flexible band may be formed of commonly available plastic or other suitable materials in one embodiment. At least one notch may be cut into the ends of the flexible band forming at least two prongs. Each of the at least two prongs may have at least one protrusion or protuberance formed into them in one embodiment. The at least one protrusion or protuberance of each of the at least two prongs may be of differing shapes and are formed into the at least two prongs at each end of the flexible band. In other embodiments the shapes of the at least one protuberance or protrusion on each of the at the least two prongs may be serrated or barbed in shape or any suitable shape but sized to fit into the at least two through holes or indentations in the clamp band slot. In other embodiments two notches are cut into the end of the disposable band forming upper, middle and lower prongs with the at least two prongs having at least one protuberance each. The at least one protuberance on each of the at middle and top prongs, in some embodiments, are sized to engage with the at least two indentations or at least two through holes in the band acceptance slot formed in the side of the disposable clamp body.

The maximum distance between the at least one protuberance on the upper prong and the at least one protuberance on the lower prong is slightly larger than the height of the band acceptance slot. Slightly larger is defined as being between about 0.01 and about 0.15 inches larger. When the flexible band is engaged into the disposable band acceptance slot the at least two prongs are depressed as they are engaged into the band acceptance slot. As the end of the flexible band is slid into the acceptance band slot the at least one protuberance engages with the at least two indentations or at least two through holes in the flexible band acceptance slots, thus connecting the flexible band with the disposable clamp.

In some embodiments, the disposable clamp can be molded or formed from plastic, metal, or both as a monolithic part. In other embodiments the clamp can be assembled from separate pieces with such pieces being pressed fit together using barbed or serrated indentations and protrusions. Such separate pieces could also be glued or welded together.

The drainage bag drape can be made of a flexible waterproof polymer and can be circular or semi-circular in shape in some embodiments. In some embodiments the drainage bag drape is clear with the upper portion of the drainage bag drape coupled to the flexible band that can be in turn attached to the disposable clamp. The disposable clamp can be attached to a surgical table rail or alternatively attached to the mount arm of the accessory mount clamp. The drainage bag drape can be “v” shaped and larger at the top than at the bottom with the bottom of the drape ending in a hole with a threaded tube attached to it. In some embodiments, the threaded tube can be attached by mechanical means or by gluing means to the hole in the bottom of the drape. The threaded tube can be attached to another threaded tube which in turn is connected to a fluid collection system which stores the fluid. In some embodiments the drape has a filter between the top of the drape and its bottom. The filter can be in the form of a mesh, which in turn can be made, for example, of cross woven thread of flexible polymer materials.

In a related aspect, a drainage bag system configured to be coupled to a medical procedural table is disclosed, which includes a drainage bag, a band attached to and supporting the drainage bag, said band having at least one protuberance positioned at least one end thereof, and at least one mounting clamp including a receiving slot configured to receive said at least one protuberance of the band. The clamp has a receiving channel that is configured to receive a procedural table accessory rail or an accessory mount arm thereof. The clamp has a threaded member configured to secure the clamp to a procedural table accessory or an accessory mount arm.

In some embodiments of the above drainage bag system, the at least one protuberance includes two protuberances each located at one of the two ends of the band and the at least one mount clamp includes two mount clamps each configured to receive one of those protuberances.

In some embodiments, the receiving slot is configured to provide at least one receptacle extending into a body of the mounting clamp for receiving said at least one protuberance so as to block the band from disengaging from the receiving slot. In some such embodiments, the at least one mounting clamp includes an upper platform, a lower platform and a clamp wall. The upper platform can include an upper protruding lip and the lower platform can include a lower protruding lip. The mounting clamp can further include at least one upper arm that extends from a proximal side of the upper platform and at least one lower arm that extends from a proximal side of the lower platform.

In some embodiments, the upper and the lower arms are substantially parallel with the surfaces of the upper and the lower platforms. In some embodiments, the lower platform comprises a threaded hole and a threaded knob to engage in the hole. The upper platform can also include a threaded hole and a threaded knob to engage in the hole.

In some embodiments, the upper platform includes a threaded hole and a threaded knob to engage a rail accessory channel formed by a lower surface of the upper platform and the protruding lip thereof, the upper surface of said lower platform and the protruding lip thereof, the arm extending from the upper and lower platforms, and the at least one side of the clamp body.

In some embodiments, the lower surface of the upper platform comprises at least one chase or channel formed therein. In some such embodiments, the at least one chase can have a width in a range from about 0.9 inches to about 1.6 inches and a height in a range of about 0.05 inches to about 0.25 inches.

The rail accessory channel is shaped and sized to receive a surgical table accessory rail or an accessory arm. In some embodiments, the rail accessory channel has a chase cut or formed therein, where the chase is configured to accept a protrusion extending out of the upper surface of an accessory mount arm. In some embodiments, the protrusion includes a fin. In some embodiments, the upper platform with the chase and the protruding lip, the lower platform and its protruding lip, the extension arms, the clamp wall, and clamp body are formed as a monolithic unit. By way of example and without limitation, such a monolithic unit can be formed of a plastic (e.g., polyethylene, polyurethane, polycarbonate, polyethylene terephthalate, or acrylonitrile-butadiene-styrene (ABS), a metal (e.g., aluminum, stainless steel, cast iron, titanium, or brass or other copper alloys), or a combination thereof.

In some embodiments, the upper platform with the chase and the protruding lip, and the lower platform and its associated protruding lip, the extension arms, the clamp wall, and the clamp body are formed as a combination of discrete parts, e.g., metal parts, plastic parts, or a combination of metal and plastic parts, that have been assembled, e.g., via press fit and/or glue and/or welding.

In some embodiments, the band of a drainage bag system according to the present teachings is configured to be received via a protrusion thereof into a pre-determined position within the clamp so as to engage with the clamp. In some embodiments, the band can have a width in a range of about 0.5 inches to about 3 inches, though other widths may also be utilized. Further, in some embodiments, the receiving channel of the clamp can have a width in a range of about 0.2 inches to about 0.45 inches and a height in a range of about 0.9 inches to about 1.6 inches.

In a related aspect, a drainage bag system configured to be coupled to a device which is configured to be coupled to a procedural table or other equipment is disclosed, which comprises a drainage bag, a band attached to and supporting the drainage bag, a mounting clamp including a receiving slot configured to receive an end of the band, where the band has at least two opposed protuberances at said end thereof configured to extend into the receiving slot and the receiving slot includes a pair of receptacles configured to receive the band protuberances for engaging the band with the mounting clamp. The clamp further includes a receiving channel to receive any of an accessory rail and an accessory mount arm of said procedural table.

In some embodiments, the clamp can include a threaded member for securing the clamp to the procedural table.

In some embodiments, the accessory rail and/or the accessory mount has a width in a range of about 0.2 inches to 0.45 inches and a height in a range of about 0.9 and 1.6 inches.

The drainage bag system can further include a device mount mechanism configured to secure the drainage bag system to any of an accessory, and a device that is coupled to the procedural table. In some embodiments, the accessory mount mechanism can include at least one arm that is shaped and sized to fit into the receiving channel.

Additional features, alone or in combination with these and any other feature(s); such as those listed above and those listed in the claims may comprise patentable material.

### BRIEF DESCRIPTION OF DRAWINGS

Drawings and figures are disclosed to aid in a description of invention and the various embodiments of the invention. The drawings and figures are attached and are referenced below.

FIGS. 1A and 1B illustrate views of a stirrup mounted onto a surgical table with a conventional drainage bag system and without such a system, respectively.

FIGS. 2A and 2B illustrate views of a disposable drainage bag system according to an embodiment of the present teachings, which is mounted onto a surgical table.

FIG. 2C is a partial perspective view of various components the disposable drainage bag system mounted on a surgical table with rail and a fluid storage system.

FIG. 2D is a close up view of the flexible band of FIG. 2C.

FIG. 3A shows a perspective of the disposable band with disposable drainage bag drape, disposable clamp and details of the reusable accessory mount clamp.

FIG. 3B is a close up view of the flexible band of FIG. 3A.

FIG. 4A shows another perspective view of the disposable band with disposable drainage bag drape, disposable clamp and details of the reusable accessory mount clamp.

FIG. 4B is a close up view of the flexible band of FIG. 4A.

FIG. 5 shows a perspective view of the disposable band with disposable drainage bag, disposable clamp and the details of reusable accessory mount clamp.

FIG. 6 shows details of a stirrup mount lug, the disposable clamp, the disposable band and the accessory mount clamp.

FIG. 7A shows another view of details of a stirrup mount lug, the disposable clamp, the disposable band and the accessory mount clamp.

FIG. 7B is a close up view of the flexible band of FIG. 7A.

FIG. 8A show details of the disposable clamps, the disposable band and the surgical table rail.

FIG. 8B is a close up view of the flexible band of FIG. 8A.

FIG. 9 illustrates details of an accessory mount mechanism coupled to a lug protruding from medical equipment in particular a stirrup mounting bracket.

FIG. 10 illustrates a cut away view of a section of the disposable clamp, the band acceptance slot, the flexible band with flexible prongs engaged in through holes within the band acceptance slot the disposable rail clamp engaged with an accessory mount arm.

FIGS. 11A and 11B illustrate cutaway sections of a disposable clamp and a rail acceptance channel of a system according to an embodiment of the present teachings.

### DETAILED DESCRIPTION

The present disclosure is generally related to a drainage bag system configured for coupling to a surgical table. Various terms are used herein in accordance with their ordinary meanings in the art. The term “about” as used herein is intended to indicate a variation of at most 10% about a numerical value. The term “surgical table” is used herein to generally refer to a medical table that is used when a medical procedure, such as a urological or gynecological procedure, is performed on a patient.

The phrase “a semi-rigid component” is defined as a member or component so formed or constructed that when a force is applied to its center the elongation of the member's non-loaded length (change in length) is greater than 0% but less than 0.10% before failure.

The phrase “a flexible component” is defined as a component or member so formed or constructed that when a force is applied to its center the elongation of the member's non-loaded length is equal to or greater than 0.10%.

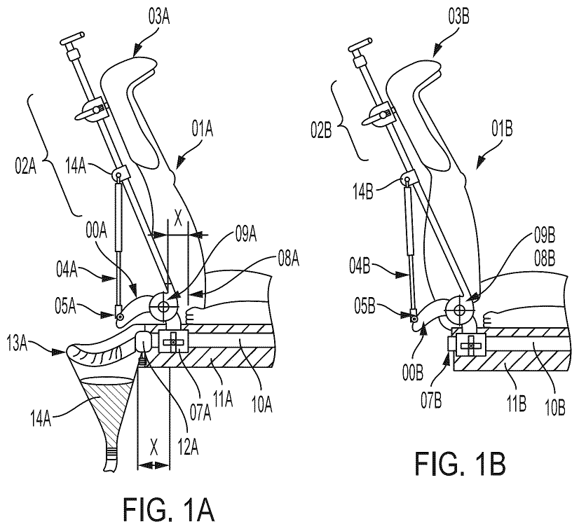

FIGS. 1A and 1B illustrate a stirrup mounted onto a surgical table without a drainage bag system and with a drainage bag system using conventional systems. A stirrup 02A of FIG. 1A is mounted to a surgical table 11A by a rail clamp 07A. Patient legs 01A are held in place by stirrup boot 03A which are supported by stirrup gas cylinder 04A attached to the stirrup at distal stirrup mount point on 14A and proximal stirrup gas cylinder mount lug 05A attached to stirrup bracket 00A. As shown in FIG. 1A, disposable clamp 12A holds flexible band 13A onto which is attached a drainage bag 14A. The mount point on the surgical table rail 10A of disposable clamp 12A can move mount point of table clamp 07A and stirrup 02A proximally down surgical table rail 10A, towards the patient's head, a distance equal to “x”. The center of rotation of the proximal stirrup locking joint 09A in FIG. 1A is offset from center of rotation of point of patient's hip 08A. This offset is caused by the mounting of disposable rail clamp 12 on distal end of surgical table rail 10A at distance “x.” FIG. 1B illustrates mounting of stirrups without using current art disposable drainage bag system. In FIG. 1B the rotation point of the patient's hip 08B and the rotation point of the proximal stirrup locking joint 09B are coterminous in the horizontal plane and thus in alignment due to rail clamp 07B being properly positioned at the distal end of the table rail 10B of surgical table 11B. Patient's legs 01B are held in the stirrup boot 03B of stirrup 02B and supported by stirrup gas cylinder 04B with distal stirrup mount points 14B and proximal stirrup gas spring mount lug 05B attached to stirrup mount bracket 00B.

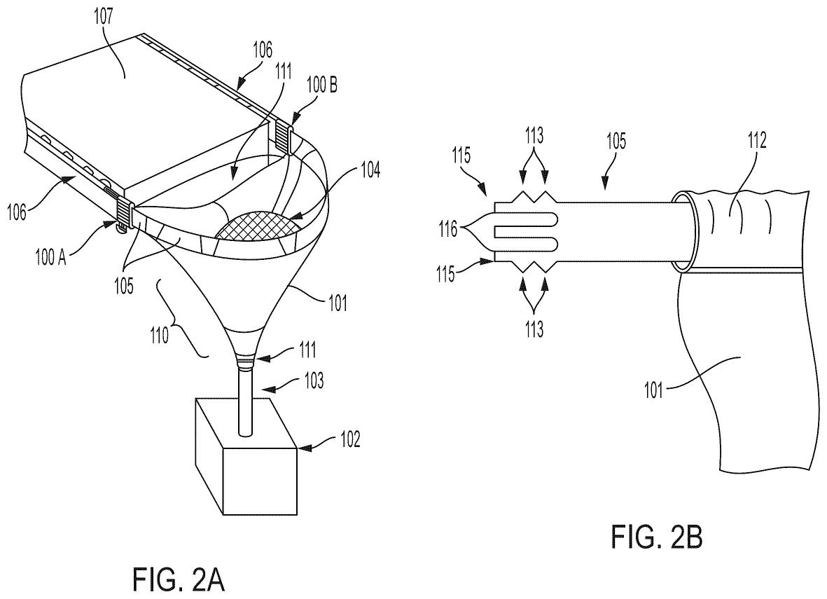

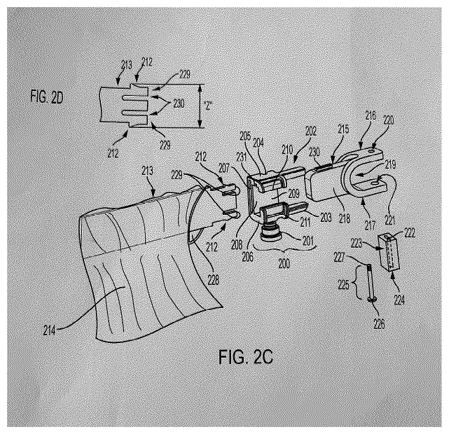

FIGS. 2A and 2B schematically illustrate a disposable drainage bag system 110 according to one embodiment of the present teachings, which is attached to a surgical table 107 side rails 106. The drainage bag system 110 includes a flexible band 105 to which the drainage bag 101 having a mesh filter 104 is attached, a first disposable clamp 100A and a second disposable clamp 100B which are shown as attached to the distal ends of surgical table side rails 106. Attached to the drainage bag 101 is a drainage tube 103 and fluid storage system 102. A drainage bag drape 110 is supported and kept open by flexible band 105 during surgery. Flexible band 105 is coupled to disposable clamps 100A and 100B which can in turn be attached to the distal ends of side rails 106 of surgical table 107. Threaded tube 103 attached to fluid reservoir 102 is engaged with and attached to flap 111 to allow fluid to flow from the drainage bag 101 into fluid storage system 102.

The illustrated drainage bag 101 is constructed from flexible sheets of thin translucent plastic materials and has a flap 111 attached to it. In this embodiment, drainage bag 101 is substantially V-shaped with the mesh filter 104 constructed out of thin flexible strips of a polymeric material laid in alternating or crossing patterns and includes a flap 111 attached at a hole 101a (not shown) at bottom of drainage bag 101 to allow fluid to drain out of the system and into a reservoir 102. Mesh filter 104 collects waste materials that may be by-products of surgical procedures and if not trapped in the mesh filter could prevent fluid from being collected in fluid reservoir 102 by clogging threaded tube 111. In other embodiments there is no mesh filter.

A plurality of different polymeric materials may be employed for constructing the drainage bag. Some examples of such polymeric materials include, without limitation, high density polyethylene, low density polyethylene polypropylene, medium density polyethylene or degradable olefinic materials.

As shown in FIG. 2B, the flexible band 105 has two notches 116 cut into its end to form upper and lower prongs 115. Upper and lower prongs 115 have two protuberances 113 formed into them. In this embodiment disposable drainage bag 101 has sleeve 112 into which flexible band 105 with prongs 115 and their associated serrated distal protuberances 113 can be inserted. It is understood that the serrated protuberances are illustrative only. In other embodiments these protuberances could be circular, serrated or barbed, rectilinear buttons, hooks, pegs or other shapes. Sleeve 112 of disposable drainage bag 101 fits around flexible band 105 and couples the disposable drainage bag 101 to the flexible band 105. Flexible band 105 supports and holds open the disposable drainage bag 101 as shown in FIG. 2A.

Flexible band 105 is flexible in the horizontal direction and semi-rigid in the vertical direction. Since it is flexible in the horizontal direction flexible band 105 is able to flex in response to clinicians moving against or contacting the band allowing the clinician to approach surgical table 107 without blocking the disposable drainage bag system 110. In this embodiment the disposable support band 105 is made from plastic which has flexible properties although in other embodiments it can be made from other suitable materials. Disposable clamps 100A and 100B are substantially equivalent being mirror images of one another. As discussed in more detail below, disposable clamps 100A and 100B can be attached to the distal end of surgical table rail 106 of surgical table 107.

FIGS. 2C and 2D show detailed aspects of embodiments of the above flexible band 105 (herein referred to for ease of description of various components as flexible band 213), disposable drainage bag 101 (which is referred to in this example as disposable drainage bag 214), disposable/mounting clamp 200 (which can correspond to one of the disposable clamps 100A/100B) and accessory mount clamp 215. In the embodiment illustrated in FIG. 2C, the disposable clamp 200 has an upper ledge 205 with upper ledge chase 231, extension stabilizer 202. Further, the disposable clamp 200 has both a lateral surface (not shown) and a medial surface 209 forming rail acceptance channel 208.

In this embodiment, under surface 205a of upper ledge 205 together with upper ledge chase 231 and stabilizer extension 202 forms the upper surface of rail acceptance channel 208 while lower ledge 206 together with stabilizer extension 203 forming the lower surface of rail acceptance channel 208. In some embodiments, a lip 210 extends down from upper ledge 205 and forms one proximal surface of rail acceptance channel 208 while a lip 211 extends from lower ledge 206 and forms another proximal surface of rail acceptance channel 208. Medial surface 209 forms proximal surface of rail acceptance channel 208. In this embodiment threaded knob 201 engages with, and fits into, threaded hole (not shown) in lower ledge 206. In other embodiments the threaded hole can be formed in the upper ledge 205.

In the embodiment illustrated in FIG. 2C, when the surgical table rail (not shown) or mount arm 218 of accessory mount clamp 215 with fin 230 is engaged into the surgical rail acceptance channel 208 of disposable clamp 200 threaded knob 201 can be engaged into and through threaded hole (not shown) in lower ledge 206. The engagement of the threaded knob 201 into threaded hole (not shown) secures the disposable clamp to the surgical table rail (not shown) or accessory mount arm 218 via its fin 230. More specifically, it can be seen in this illustration that the fin 230 fits into upper ledge chase 231 when threaded knob is engaged into and through threaded hole in lower ledge 206 (not shown). After a surgical procedure is completed, threaded knob 201 can be disengaged from the hole in lower ledge 206 allowing disposable clamp 200 to disengage from mount arm 218 with fin 230 (or surgical table rail) and be removed.

In this embodiment when surgical table rail (not shown) or mount arm 218 with fin 230 is engaged into rail acceptance channel 208 of the disposable clamp with upper ledge chase 231 and stabilizer extensions 202 and 203, the disposable clamp 200 is protected from any torsion or rotational forces that may be encountered. In this embodiment disposable clamp 200 has flexible band acceptance slot 207 formed into one of its sides. Two through holes are formed into band acceptance slot 208 with one on the upper side of band acceptance slot 207 (visible) and one on its lower side (not visible in this illustration).

However, in other embodiments, more than two through holes may be formed into band acceptance slot 207 or only one through hole may be so formed in yet other embodiments. It should also be understood that although through holes are shown in this embodiment in other embodiments indentations can be formed in band acceptance slot 207. In this embodiment through hole 204 in band acceptance slot 207 is shown as being rectilinear but it is understood that through hole 204 can be of shapes differing from that shown in FIG. 2C. Alternatively, as noted above, in other embodiments rather than one or more through holes there can be indentations formed in band acceptance slot 207.

In this embodiment disposable flexible band 213 is fitted into sleeve 228 of disposable drainage bag 214 as shown in FIG. 2C. Flexible band 213 has notches 230 cut into its ends forming insertion prongs 229 with serrated protuberances 212. In this embodiment the maximum distance “Z” between serrated protuberances 212 is slightly larger than the height of band acceptance slot 207. As used herein, the phrase “slightly larger” is defined as being between 0.01 inches to 0.15 inches larger. As the end of flexible band 213 is engaged into band acceptance slot 207 protuberances 212 are slightly depressed such that when protuberances 212 engage with through holes 204 the tension on them is released, thereby engaging protuberances 212 in the through holes 204 and connecting flexible band 213 with disposable clamp 200. In other embodiments through holes 204 might be indentations sized to accept protuberances 212.

One embodiment of accessory mount clamp 215 as shown in FIG. 2C includes accessory mount clamp 215 comprising an upper arm 216 with hole 220, a lower arm 217 with threaded hole 221, semi-circular section 219 with mount arm 218 with fin 230 plus spacer 223 with through hole 224. Connecting rod 225 includes a threaded end 227 with a flat head on end 226. Connecting rod 225 can be pushed into and through hole 220 that is formed in the upper arm 216 and passed into hole 222 of spacer 223 and out through hole 224 and be engaged into threaded hole 221 in lower arm 217 thus affixing spacer 223 to accessory mount clamp 215. In this embodiment, accessory mount clamp 215 is formed as a monolithic structure out of plastic or metal although it is understood that each of the component parts could be assembled by gluing, press fitting or welding these parts together in any combination. Spacer 224 in this embodiment is shown as a rectilinear block although in other embodiments it could be square, circular, semi-circular or a “t” shaped monolith. The rail access channel 208 with extension stabilizers 202 and 203 of disposable clamp 200 can be engaged with mount arm 218 while mount arm fin 230 can be engaged into upper ledge chase 231 of accessory mount clamp 215.

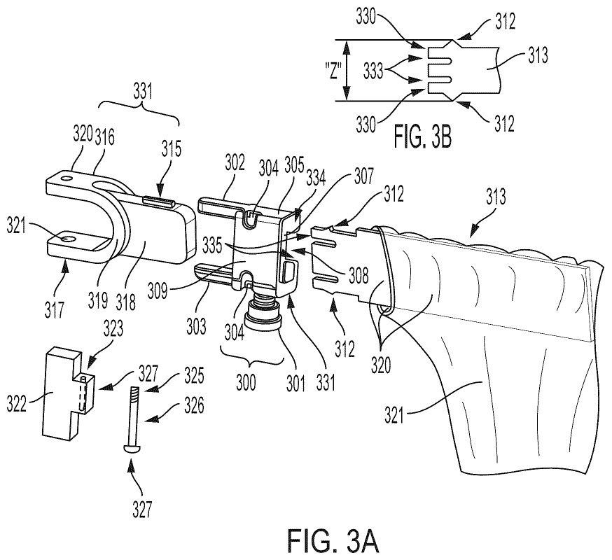

FIGS. 3A and 3B show another perspective view of an embodiment of a flexible band 313, disposable drainage bag 321, disposable clamp 300, accessory mount clamp 316, spacer 322 and rod 326. Flexible band 313 is engaged into sleeve 320 of drainage bag 321. In this embodiment disposable clamp 300 has upper ledge 305. The upper ledge 305, extension stabilizer 302 along with the proximal surface of clamp wall 309 form the distal surface of rail acceptance channel 308. In this embodiment, an upper ledge chase 334 is formed into an under surface of upper ledge 305.

Upper ledge 305 stabilizer extension 302 forms the upper surface of rail acceptance channel 308 while lower ledge 331 with stabilizer extension 303 forms the lower surface of rail acceptance channel 308. In some embodiments threaded knob 301, engages with, and fits into, a threaded hole (not shown) in lower ledge 331. When engaged into rail acceptance channel 308, threaded knob 301 engages accessory mount arm 318 via fin 315 within rail acceptance channel 308. In this embodiment flexible band 313 has two notches 333 cut into its end forming upper and lower prongs 300 with protuberances 312. The maximum distance between protuberances 312 of “Z” is slightly larger than the height of flexible band acceptance slot 307 of disposable clamp 300. As noted above, the phrase “slightly larger” as used herein is intended to indicate about 0.01 inches to about 0.15 inches larger. When the end of flexible band 313 is engaged into band acceptance slot 307 of disposable clamp 300, protuberances 312 are slightly depressed creating tension such that the engagement of protuberances 312 with holes 304 in the band acceptable slot 307 secures the disposable clamp 300 to flexible band 313.

FIGS. 3A and 3B show an embodiment of accessory mount clamp 331 comprising an upper arm 316 with hole 320, a lower arm 317 with threaded hole 321, a semi-circular section 319 with mount arm 318 with fin 315 plus spacer 322 with through hole 323. In this embodiment although spacer 322, with flange 324, is shown as a “t” shaped part it is understood that it could also be square, rectilinear, spherical or semi-spherical or other appropriate shapes. Connection rod 326 includes a threaded end 325 with a flat head on end 327. Connection rod 326 can be pushed into and through hole 320 in upper arm 316 and passed into through hole 323 in flange 324 of spacer 322 and out through the hole to be engaged into threaded hole 221 of lower arm 217, thereby engaging spacer 323 with accessory mount clamp 331.

In this embodiment the “u” shaped portion of accessory mount clamp 331 is formed as a monolithic structure out of plastic or metal or both although in some embodiments, the mount clamp 331 can be formed as a plurality of discrete component parts that are assembled together, e.g., via gluing, press fitting or welding. Rail access channel 308 with extension stabilizers 302 and 303 and upper ledge chase 334 of disposable clamp 300 can be engaged with mount arm 318 of accessory mount clamp 331.

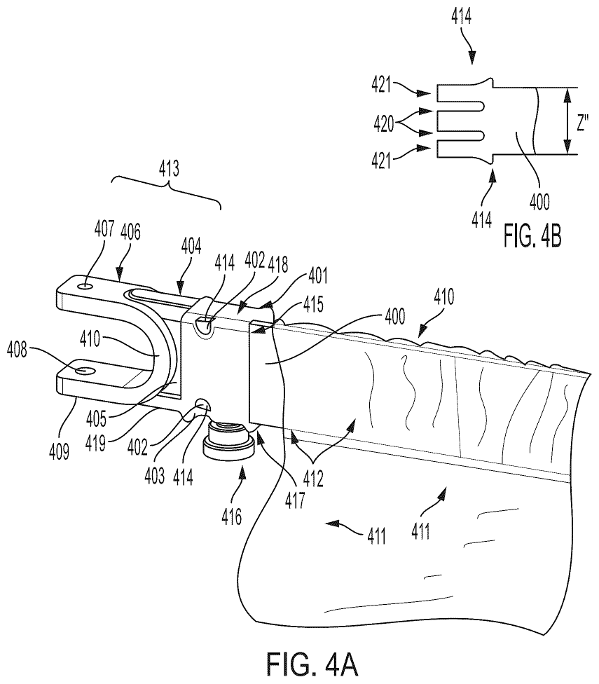

FIGS. 4A and 4B illustrate an embodiment of a flexible band 400 fitted into sleeve 412 of a disposable drainage bag 411 engaged into disposable clamp 401, which in turn is connected with accessory mount clamp 413. In this embodiment, flexible band 400 is fully engaged into flexible band acceptance slot 415. Flexible band has notches 420 cut into its end to form upper and lower prongs 421. Protuberances 414 are formed on prongs 421. Distance “Z” between protuberances 414 is slightly larger than the height of band acceptance slot 415 of disposable clamp 418. As noted above, the phrase “slightly larger” as used herein indicates between about 0.10 and about 0.15 inches larger.

When the end of flexible band 400 is engaged into band acceptance slot 415, protuberances 414 are slightly depressed creating tension such that when protuberances 414 are engaged into through holes 414 flexible band 411 is thus secured to disposable clamp 418. An accessory mount clamp 413 includes an upper arm 406 with hole 407, a lower arm 409 with threaded hole 408 and a semicircular section 410 with extension arm 405. Mount arm 405 extends at right angles from semi-circular section 410. Rail acceptance channel of disposable clamp 401 formed by upper ledge 418 with stabilizer extension 404 and lower ledge 417 with stabilizer extension 419 can be engaged with mount arm 405 of accessory mount clamp 413.

Threaded knob 416 can be engaged into threaded hole in lower ledge 417 of disposable clamp 410, thereby securing mount arm 405 to disposable clamp 401 which in turn secures accessory mount clamp 413 to disposable clamp 401.

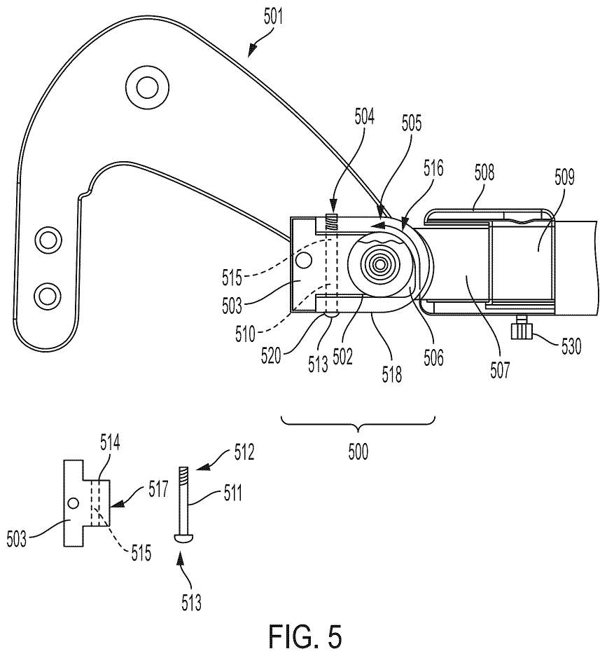

FIG. 5 illustrates an embodiment of the present teachings in which a mount 500 is attached to stirrup gas cylinder proximal mount lug 502 as well as to disposable clamp 509.

Stirrup bracket 501, which mounts stirrup (not shown) to surgical table (not shown) has stirrup gas cylinder proximal mount lug 502 attached to it. Accessory mount clamp 516 includes an upper arm 505 with threaded hole 504, a semi-circular section 506 with mount arm 507 at right angles, and a lower arm 518 with hole 510. By passing rod 511 with threaded end 512 and flat head end 513 through hole 520 in lower arm 518 of accessory mount clamp 516 and into and through hole 514 of spacer 503 so that threaded end of rod 511 engages with threaded hole 504 of upper arm 505, accessory mount clamp 516 and spacer 503 can be connected around stirrup gas cylinder proximal mount lug 502.

With accessory mount clamp 516 connected to spacer 503, the surfaces of upper arm 505, semi-circular section 506, lower arm 518 and spacer 503 are snugged up against the surface of stirrup gas spring proximal mount lug 502. Disposable clamp 509 can be engaged with mount arm 507 securing it to stirrup gas cylinder mount lug 502.

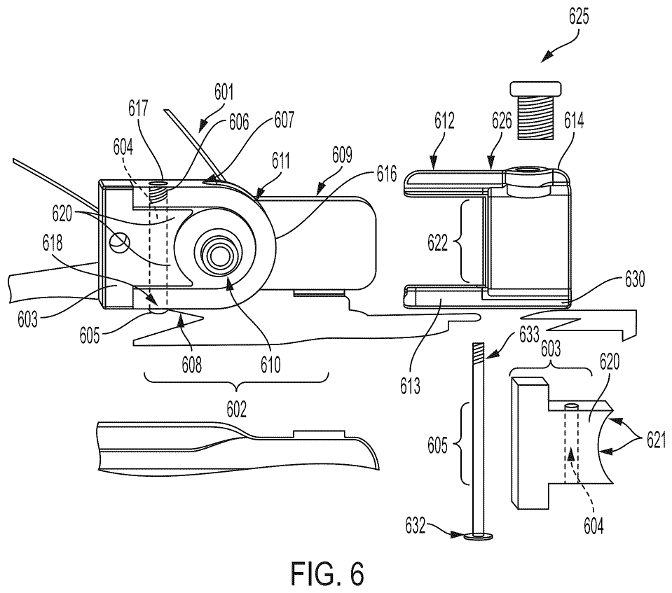

FIG. 6 illustrates an expanded view of an embodiment of a mount 602. The mount 602 includes a “t” shaped spacer 603 with flange 620 with semi-circular cut-out 621, attachment rod 605 with threaded end 633 and flat head end 632 and through hole 604 plus accessory mount clamp 611, which includes an upper arm 607 with threaded hole 617 and a lower arm 608 with hole 604. In this embodiment, spacer 603 flange 620 with semi-circular cutout is shaped so that its surface snugs up against the surface of mount lug 610., which is attached to stirrup bracket 601, which mounts stirrup (not shown) to surgical table (not shown).

In this embodiment, spacer 603 is shown as a “t” shaped monolith however, in other embodiments, spacer 603 could be square or rectilinear while flange 620 could be rectilinear, round or obloid in shape. Accessory mount clamp 611 includes a top arm 607 with a threaded hole 617, a semi-circular section 616 and a lower arm 608 with hole 618. In this embodiment accessory mount clamp 611 is shown as a monolith but it could be made of a number of discrete component parts that could be pressed fit, welded or glued together. Spacer 603 could be made of plastic or metal or combination of these materials, such as those listed above in connection with the previous embodiments.

Spacer 603 has through hole 604 in flange 620 with semi-circular cut out 621 allowing the surface of the flange 620 to mate closely and intimately with the proximal surface of gas cylinder proximal mount lug 602. Accessory mount clamp 611 also includes a mount arm 609. Disposable clamp 630 has rail acceptance channel 622 formed by upper and lower stabilizer extensions 612 and 613 and the lower surface of upper ledge 626 and upper surface of lower ledge 630, respectively. In this embodiment, rail acceptance channel 622 of disposable clamp 630 can be engaged onto mount arm 609 of accessory mount clamp 611. Threaded hole 614 is formed in top ledge of 626 into which threaded knob 625 can be engaged. Mount arm 609 of accessory mount 602 can be engaged into rail clamp acceptance channel 622 of disposable clamp 630. When threaded knob 625 is engaged in threaded hole 614, disposable clamp 630 is secured to the mount arm of reusable mount 602. In this embodiment, threaded hole 614 is formed in top ledge 626, but in other embodiments, it could be formed in a bottom ledge of disposable clamp 630.

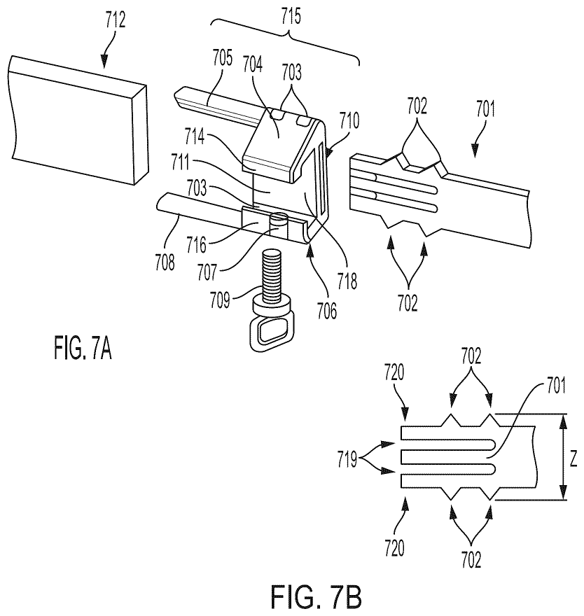

FIGS. 7A and 7B provide details of connections between surgical table side rail 712, disposable clamp 715 and flexible band 701. Disposable clamp 715 has upper ledge 704 with lip 714, lower ledge 706 with lip 716, proximal clamp wall 718, an upper stabilizer extension 705, a lower stabilizer extension 708 and band acceptance slot 710 with through holes 703. This embodiment shows four through holes but other embodiments could have at least two through holes. Yet other embodiments could have at least two indentations instead of through holes. Lower ledge 706 has threaded hole 707 into which threaded knob 709 can be engaged. Rail access channel 711 of disposable clamp 715 is formed from proximal clamp wall 718, lower surface of upper ledge 704, upper surface of lower ledge 706, upper lip 714, lower lip 716, upper extension 705, and lower extension 708.

Surgical table rail 712 can be engaged into rail acceptance channel 711 and threaded knob 709 can be engaged into and through threaded hole 707 securing disposable clamp 715 to surgical table rail 712. Flexible band 701 has notches 719 formed or cut into its end to form prongs 720. Prongs 720 have protuberances 702. This embodiment shows two protuberances formed on each of prongs 720 but other embodiments may have one protuberance on each prong while yet other embodiments may have three or more protuberances. Distance “z” between prongs 720 is slightly larger than the height of band acceptance slot 710. The phrase “slightly larger” is defined as between 0.10 to 0.15 inches larger. When the end of flexible band 701 is engaged into band acceptance slot 710, protuberances 702 are slightly depressed. When protuberances 702 are engaged into holes 703 of disposable clamp 715, flexible band 701 is secured to disposable clamp 715.

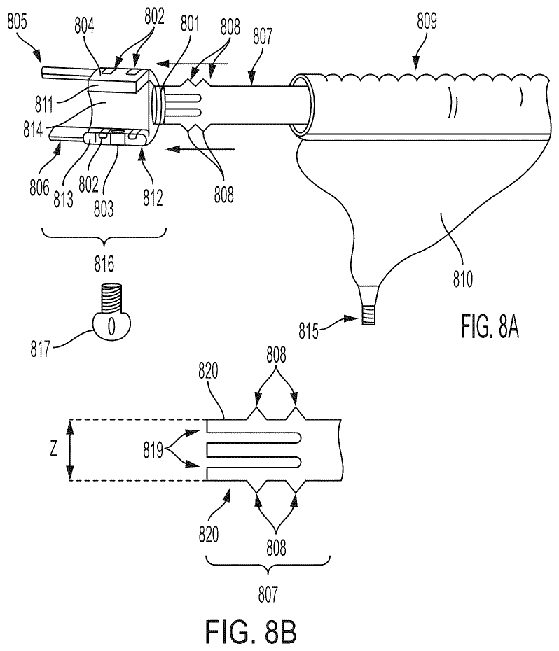

FIGS. 8A and 8B show details of flexible band 807, disposable drainage bag 810 with sleeve 809 and disposable clamp 816. Disposable clamp 816 has an upper ledge 804 with lip 811, a lower ledge 812 with lip 813, a proximal clamp wall 814, an upper stabilizer extension 805, a lower stabilizer extension 806, and a band acceptance slot 801 with through holes 802. This embodiment shows four through holes but other embodiments could have at least two through holes. Other embodiments could have at least two indentations instead of through holes. The lower ledge 812 has threaded hole 803 into which threaded knob 817 can be engaged. The end of flexible band 807 has two notches 819 cut into it forming prongs 820 with protuberances 808. This embodiment shows two protuberances 808 formed into each prong 820 but in other embodiments prong 820 may have one or more protuberances 808. The distance “z” is slightly larger than the height of band acceptance slot 801. As noted above, the phrase “slightly larger” is intended to indicate that the distance “z” is larger than the height of band acceptance slot 801 by an amount in a range of about 0.10 to 0.15 inches.

When the end of the flexible band 820 is engaged into band acceptance slot 801 protuberances 808 are slightly depressed such that when protuberances 808 are engaged into holes 802 of disposable clamp 816 flexible band 829 is secured to disposable clamp 816. Disposable drainage bag 810 has sleeve 809 which can be fitted onto flexible band 807. Disposable drainage bag 810 and sleeve 809 can be made of clear plastic. Threaded tube 815 is attached to the bottom of drainage bag 810 to allow fluid to flow into fluid collection reservoir (not shown).

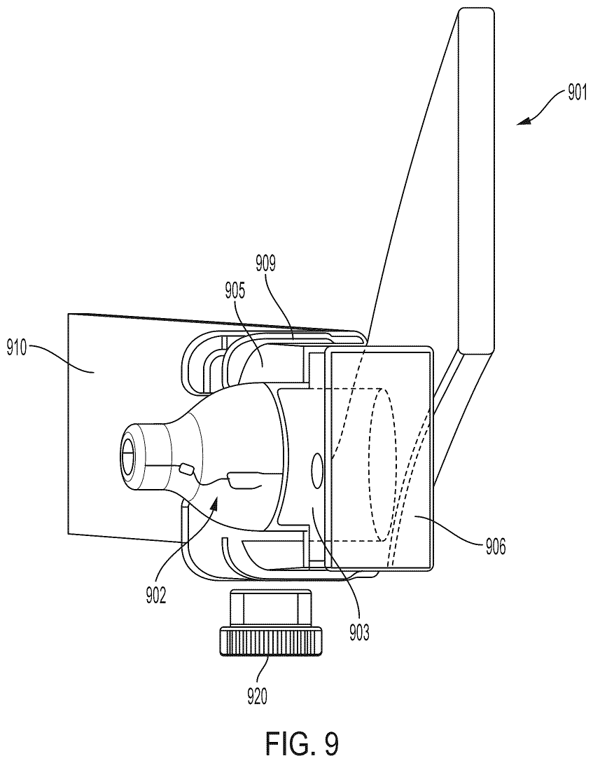

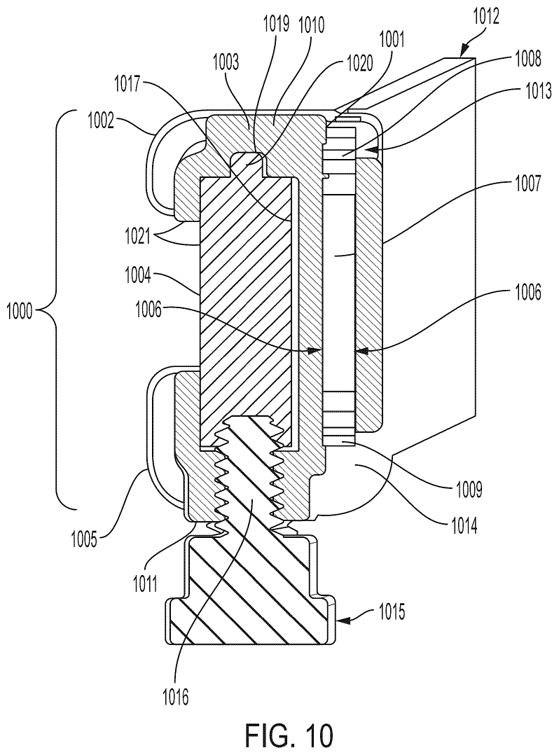

FIG. 9 illustrates some aspects of an embodiment of an accessory mount mechanism attached to a protrusion extending from surgical equipment in particular a lithotomy stirrup. This includes the distal surface of flange 903 of spacer 906 which fits snuggly against the proximal surface of stirrup gas cylinder mount lug 902, which is attached to stirrup mount bracket 901. The proximal surface of accessory mount clamp 905 fits snuggly against the upper, lower and distal surface of stirrup cylinder gas lug 902. Flexible band 910 is attached to disposable clamp 909 which in turn is attached to the accessory mount mechanism mount arm (not shown).

FIG. 10 shows a cut-away view of the disposable clamp 1000 with upper ledge 1010 and lower ledge 1011, lower lip 1005 and upper lip 1002, clamp wall 1001 with inner wall 1017, band acceptance slot 1006 with flexible band 1012 engaged in said slot with lower prong protuberance 1009 and upper prong protuberance 1013 of flexible band 1012 engaged in through holes 1014 and 1013. Rail acceptance channel 1021 is formed by an upper ledge 1010, a lower ledge 1011, a lower lip 1005, an upper lip 1002 and an inner wall 1017 of disposable clamp 1000. Upper ledge chase 1019 is cut or formed into the lower surface of upper ledge 1010. Accessory mount arm 1004 is shown engaged in rail acceptance channel while fin 1020 is engaged into upper ledge chase 1019. Threaded knob 1015 is engaged into and through threaded hole 1016 of lower ledge 1011 securing mount arm 1004 to disposable clamp 1000.

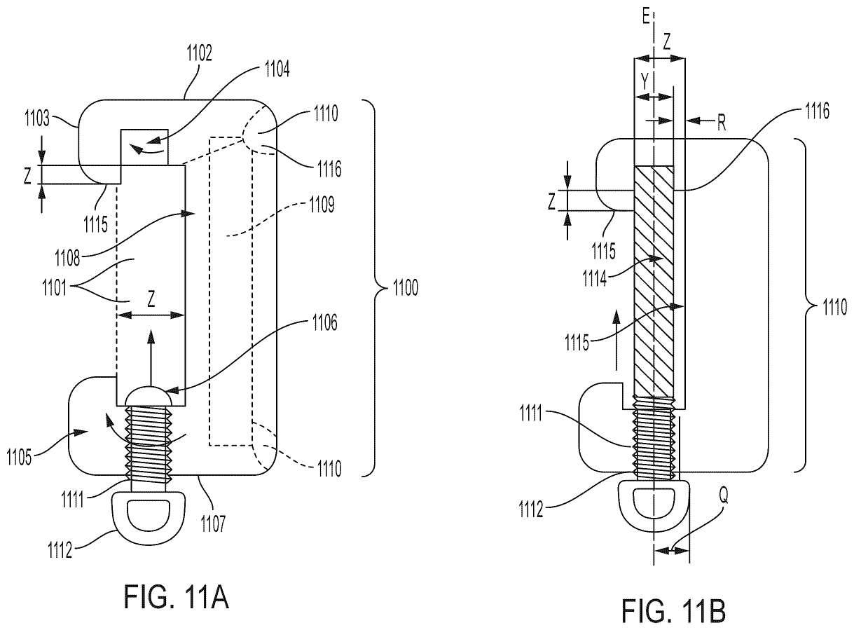

FIGS. 11A and 11B illustrate one cutaway view of one embodiment of the disposable clamp 1100 with upper ledge 1102, lower ledge 1107, upper lip protrusion 1103 with engagement surface 1115, lower lip protrusion 1105, upper ledge chase 1104, proximal clamp wall 1115 with notch 1116 making up rail acceptance channel 1101. In one embodiment, rail acceptance channel 1101 has width “Z”, e.g., about 0.4 inches. In some embodiments the upper surface of notch 1116 is at a distance “N” from lower edge of engagement surface 1115, where in some embodiments N may be equal to about 0.125 inches. Disposable clamp 1100 has band acceptance slot 1109 with slot through holes 1110, and threaded hole 1111 in lower ledge 1107 with threaded knob 1112. In this embodiment center line “E” of threaded hole 1111 in lower ledge 1107 is offset from center line “E” of disposable clamp 1110 by a distance “Q.” Width of rail 1114 less than the width of rail acceptance channel 1101 by an amount “R,” where in some embodiments “R” may be equal to about 0.15 inches and width “Y” of chase 1114 formed into lower surface of upper ledge 1102 may be equal to about 0.25 inches. When fully engaged into threaded hole 1111 threaded knob 1112 pushes up on table rail 1114 with width “Y” into chase 1104.

Although certain illustrative embodiments are described in detail above; many embodiments, variations and modifications are possible that would be within the scope and spirit of this disclosure as described herein and as defined the claims that follow.

1. A drainage bag system configured to be coupled to a medical procedural table, comprising:
a drainage bag,
a band attached to and supporting the drainage bag, said band having at least one protuberance positioned at least one end of the band, and
at least one mounting clamp including a receiving slot configured to receive said at least one protuberance of the band,
wherein the at least one mounting clamp has a rail acceptance channel configured to receive a surgical table accessory rail or an accessory mount arm of an accessory mount clamp, wherein the at least one mounting clamp has a threaded member configured to secure the at least one mounting clamp to the surgical table accessory rail or to the accessory mount clamp, wherein the at least one mounting clamp includes an upper platform, a lower platform and a clamp wall,
wherein the upper platform includes an upper protruding lip and the lower platform includes a lower protruding lip,
wherein the accessory mount clamp comprises at least one upper arm, at least one lower arm, wherein said upper and lower arms are substantially parallel with a top surface of the upper platform and a bottom surface of the lower platform, respectively,
wherein said lower platform comprises a threaded hole and the threaded member includes a threaded knob to engage in the threaded hole, and
wherein the rail acceptance channel is formed by a lower surface of said upper platform, the upper protruding lip, an upper surface said lower platform and the lower protruding lip.

2. (canceled)

3. The drainage bag system of claim 1, wherein said receiving slot includes at least one through hole for receiving said at least one protuberance so as to block the band from disengaging from the receiving slot.

4. (canceled)

5. (canceled)

6. (canceled)

7. (canceled)

8. (canceled)

9. (canceled)

10. (canceled)

11. The drainage bag system of claim 1, wherein the lower surface of the said upper platform comprises at least one chase or channel formed therein.

12. The drainage bag system of claim 11, wherein said at least one chase has a width in a range from about 0.9 inches to about 1.6 inches.

13. The drainage bag system of claim 12, wherein said at least one chase has a height in a range of about 0.05 inches to about 0.25 inches.

14. The drainage bag system of claim 1, wherein the rail acceptance channel is configured to be shaped and sized to receive a surgical table accessory rail or an accessory mount arm.

15. The drainage bag system of claim 14, wherein the rail acceptance channel has a chase cut or formed therein, wherein the chase is configured to accept a protrusion extending out of the upper surface of the accessory mount arm.

16. The drainage bag system of claim 15, wherein said protrusion comprises a fin.

17. The drainage bag system of claim 15, wherein the upper platform and the upper protruding lip, and lower platform and the lower protruding lip, and the clamp wall are formed as a monolithic unit.

18. The drainage bag system of claim 17, wherein said monolithic unit is formed of any of a plastic, a metal and a combination thereof.

19. The drainage bag system of claim 18, wherein said plastic comprises any of high-density polyethylene, low density polyethylene, polypropylene, medium density polyethylene or a degradable olefinic material.

20. The drainage bag system of claim 18, wherein said metal comprises any of aluminum, stainless steel, cast iron, titanium, or brass or other copper alloys.

21. The drainage bag system of claim 1, wherein the upper platform and upper protruding lip, lower platform and lower protruding lip, and the clamp wall are formed as a combination of metal parts that may be glued or press fit together.

22. The drainage bag system of claim 1, wherein the upper platform and upper protruding lip, lower platform and lower protruding lip, and the clamp wall are formed out of a combination of plastic parts that may be welded, glued or press fit together.

23. The drainage bag system of claim 1, wherein the band is configured to be received via a protrusion formed on the band into a pre-determined position within the at least one mounting clamp so as to engage with the at least one mounting clamp.

24. The drainage bag system of claim 1, wherein said band has a width in a range of about 0.5 inches to about 3 inches.

25. The drainage bag system of claim 1, wherein said rail acceptance channel of the at least one mounting clamp has a width in a range of about 0.2 inches to about 0.45 inches and a height in a range of about 0.9 inches to about 1.6 inches.

26. (canceled)

27. (canceled)

28. (canceled)

29. (canceled)

30. (canceled)
